(19) United States
(12) Patent Application Publication
(10) Pub. No.: US 2022/0143602 A1
(43) Pub. Date: May 12, 2022

(54) DIVISIBLE MULTI-WELL PLATES

(71) Applicant: ABGENE LIMITED, Carlsbad, CA (US)

(72) Inventors: Daniel SIMS, Ashford (GB); Peter PRYCE, East Sussex (GB); Gabor NAGY, Chester Le Street (GB)

(21) Appl. No.: 17/433,504

(22) PCT Filed: Mar. 13, 2020
(86) PCT No.: PCT/GB2020/050647

§ 371 Date: Aug. 24, 2021

Related U.S. Application Data
(60) Provisional application No. 62/818,669, filed on Mar. 14, 2019.

(51) Int. Cl. B01L 3/00

(52) U.S. Cl. CPC B01L 3/50855; B01L 2300/12; B01L 2300/0829

(57) ABSTRACT

The present disclosure describes multi-well plates comprising a frame made of a first material having an array of a plurality of holes arranged in a columnar pattern on the top surface and an array of segmented breaking features arranged in a linear columnar pattern which alternates with the columnar pattern of the plurality of holes, and a plurality of tubes/wells made of a second material placed into the plurality of holes, wherein the multi-well plate is manually divisible along the segmented breaking features into one or more columns of multi-well/tube strips. Manually dividing multi-well plate of the disclosure produces multi-well strips or segments that have smooth edges, thereby reducing injuries and glove tear incidences.

### CROSS-REFERENCE TO RELATED APPLICATIONS

This application is a 371 U.S. National Phase Application of PCT/GB2020/050647 filed Mar. 13, 2020 (now published as WO2020/183195), which application claims the benefit under 35 U.S.C. § 119(e) of U.S. Provisional Application No. 62/818,669 filed Mar. 14, 2019. The entire contents of the aforementioned applications are incorporated by reference herein.

### TECHNICAL FIELD

The present disclosure relates broadly, but not exclusively, to multi-well plates that can be manually divided into strips. Manually divided tube strips of the disclosure have smooth edges that prevent injury or glove tearing to a user.

### BACKGROUND

Reagents for biological and chemical methods are often supplied to users, pre-aliquoted in multi-well plates, such as 96-well plates. Often these reagents are supplied as lyophilized and/or frozen formulations. In a typical multi-well plate configuration, for a frozen reagent, a user would need to thaw the entire multi-well plate and either use all the reagents at once or discard unused sections of the plate. Reagents can alternatively be supplied pre-aliquoted in strips of tubes (such as 8-tube strips), but this is not suited to the manufacturing environment because strip tubes need to be manually loaded into automated filling equipment and manually sealed with cap strips. Multi-well plates on the other hand have been standardized for automatic reagent dispensing and sealing.

Some existing multi-well plates are divisible into tube strips or segmented plates. However, these are manufactured entirely from polypropylene or similar soft material, which, while suitable for reactions that require temperature changes such as the polymerase chain reaction (PCR) are not suitable for automation systems and manufacturing processes. Due to characteristics of the material, these tube strips or segmented plates are hard to handle by humans or automation systems in a manufacturing environment. For example, the softness of polypropylene makes the plates difficult to grip by automated handlers and robotic devices. Grippers used by automation systems could warp, deform or crush the plate due the flexible, semi-rigid nature of polypropylene material.

Some existing polypropylene divisible plates require scissors, cutters or other cutting tools to be divided into sections.

In addition, some existing divisible multi-well plates are only divisible into segmented plates, i.e., are divisible into 24-well or 32-well segments and not into single 8-tube strip. This again results in wastage of unused reagents.

There are some divisible multi-well plates that have a frame made of a firm material and wells made of a soft material that are suitable for automated handling. However, in some cases, such multi-well plates require a tool such as a scissor or a cutter, for breaking the multi-well plate apart into tube strips. In other cases, such multi-well plates, are manually divisible but they are only divisible into segments (such as 32 well blocks) and are not amenable for dividing into single strips (such as either tube strips). In yet other cases, in existing manually divisible multi-well plates, which can be divided into single strips, the dividing process leaves sharp edges that cause glove tears or injury to users.

A need therefore exists for multi-well plates that are manually divisible, without use of any cutting tool, in single tube strip formats that that address at least some of the problems described above.

### SUMMARY

One aspect of the present disclosure provides a multi-well plate that is manually divisible into strips of wells or tubes that are compatible with reagent dispensing by human or automation systems in manufacturing environments. One aspect of the present disclosure provides a multi-well plate that is manually divisible into tube strips or well strips (such as but not limited to 8-well or 8-tube strips) or into segments comprising at least one or more tube strips (segments of 8-tube or 8-well strips such as segments with 16, 24, 32, 40, etc. tubes). The terms wells or tubes are used alternatively in this application.

In some embodiments, the disclosure provides a multi-well plate comprising a frame portion made of a first material, wherein the frame has a planar top surface having an array of a plurality of holes arranged in a columnar pattern (forming a columnar pattern of a plurality of holes) on the top surface, the frame further comprising an array of breaking features arranged in a columnar pattern (forming a columnar pattern of breaking features) alternating with the columnar pattern of the plurality of holes, and a plurality of tubes or wells placed into the plurality of holes on the top surface of the frame, wherein the tubes or wells are made of a second material and wherein the multi-well plate is manually divisible along a center section of the breaking features into one or more columns of multi-well strips. In some embodiments, the breaking features are segmented breaking features.

In some embodiments, multi-well plates of the disclosure, are manually divisible along the center of the segmented breaking features into a plurality of single columns of multi-well strips. Multi-well strips can also be divided into segments comprising at least two columns of multi-well strips.

In some embodiments, segmented breaking features of the multi-well plates of the disclosure, comprise an array of petal shaped cuts that extend from the top surface of the frame through a bottom surface of the frame and a series of undercuts on a bottom surface of the frame, arranged such that the undercuts alternate with the petal shaped cuts. In some embodiments, the undercuts are V-shaped undercuts.

In some embodiments of a multi-well plate of the disclosure, petal shaped cuttings on the top surface of the frame combined with an undercut on the bottom surface of the frame creates eight small breaking sections or breaking features on each side of a column of multi-well plates. The length and thickness of each breaking section is the same across the frame. This pattern of breaking sections results in a segmented breaking pattern.

Advantageously, the segmented breaking pattern/feature, in contrast to continuous break lines that are present in some existing multi-well plates, provides one or more of the following improvements: eliminates the need of a tool for dividing the multi-well plate, and/or allows for manual tearing of the multi-well plate into strips/segments, and/or radius of petal shape and the localized breaking minimizes sharp edges after breaking and/or reduces sharp, jagged edges after breaking, and/or results in smooth edges and/or results in reducing the likelihood to cause glove tears or user hand and finger injuries.

The breakable feature design of the present disclosure prevents end user's gloves from tearing and/or prevents injury such as cuts and nicks to fingers and hands of a user. In some embodiments, multi-well plates of the disclosure, when manually divided into strips or segments of tubes or wells, provide or result in smooth edges, and/or minimize or reduce sharp edges and/or significantly reduce sharp or jagged edges. In some embodiments, manually dividing a multi-well plate of the disclosure results in one or more columns of multi-well strips that have no sharp edges. Accordingly, multi-well plates of the disclosure can be divided into strips or segments with reduced injury and/or no injury to a user. Multi-well plates of the disclosure can be divided into strips or segments with reduced or no glove tearing incidences.

In some embodiments, multi-well plates of the disclosure comprise a frame made of a first rigid material and the wells are made of a second softer material. In some embodiments, the first rigid material comprising the frame is polycarbonate, or an amorphous plastic and a partially crystalline heavily filled plastic and combinations thereof. In some embodiments, the second softer material comprising the wells is polypropylene, silicone, LSR (Liquid silicone rubber), or made of a soft plastic and partially crystalline plastic and combinations thereof. In some embodiments, the first rigid material comprising the frame is polycarbonate and the second softer material comprising the wells is polypropylene.

In some embodiments, wells or tubes of a multi-well plate of the disclosure, are directly molded into the frame utilizing form locking connections. In exemplary embodiments, molding and mechanical interlocking of parts on the underside of the frame forms the locking connections to join the wells or tubes to the frame.

In some embodiments, multi-well plates comprise 24, 48, 96, 384 or 1536 or more wells. In some embodiments, multi-well plates comprise 96 wells. In some embodiments, a multi-well plate of the disclosure can be manually divided into one or more columns of 8 tube strips or into segments comprising at least one column of 8-tube strips.

In some embodiments, multi-well plates of the disclosure, are compatible with one or more of the following including but not limited to: automation, manufacturing processes, lyophilization procedures, filling of reagents, freezing reagents, use in thermal cycling instruments, use in microbial processes and the like.

Multi-well plates of the disclosure and the corresponding 8-tube strips and/or segments comprising at least two or more 8-tube strips are compatible with several commercially available thermal cycler instruments.

Multi-well plates of the disclosure, in some embodiments, can be sealed to enclose reagents. Seals can comprise but are not limited to one or more of peelable foil, clear seals, clear caps, optical seals, optical caps, domed caps, flat caps and the like. Frames, skirts and wells of the multi-well plates of the disclosure can additional comprise features that fit and interface with a variety of automation devices and can have features to enable robotic gripping.

In some embodiments, a skirt feature is located extending from the edges along the frame of a multi-well plate either on parts of the frame or all along the frame. In some embodiments, multi-well plates of the disclosure, are of a standard profile design wherein the height of the skirt is of standard height, which allows tubes/wells of standard height to fit into the frame. In such embodiments, the skirt has an extended lip (sometimes referred to in the art as the SBS (Society for Biomolecular Screening) lip) that that wraps all around the perimeter of the skirt and plate. Such plates, in some embodiments, allow standard height multi-well tubes to be associated with the frame.

In some embodiments, multi-well plates of the disclosure, are low profile in design, wherein the height of the skirt is shorter than the standard height, which allows low profile tubes/wells to fit into the frame. In such embodiments, the skirt lip (referred to as SBS lip) is only provided at the four corners of the skirt of a multi-well plate.

Both standard profile and low profile multi-well plates of the disclosure and their divisible strips and segments are useful in commercially available thermocycler instruments such as those used to perform polymerase chain reaction (PCR) and quantitative PCR (qPCR). Another advantage of the multi-well plates of the present disclosure is therefore provision of a consumable multi-well plate with either pre-aliquoted, frozen or lyophilized reagents that are usable with common thermocycler instruments available in the market. One strip or segments with more than one strip can be used variously with different thermal cyclers.

These and other features of the present teachings will become more apparent from the detailed description in sections below.

### BRIEF DESCRIPTION OF THE DRAWINGS

One or more embodiments of the present disclosure may be better understood in reference to one or more the drawings below. The skilled artisan will understand that the drawings, described below, are for illustration purposes only. The drawings are not intended to limit the scope of the present teachings in any way.

FIG. 1 shows a perspective view of a frame portion of a multi-well plate according to an example embodiment.

FIG. 2 shows front view of the frame of FIG. 1, with the dotted circled portion depicting an undercut on the bottom surface of the frame.

FIG. 3 shows an exploded view of the dotted circled portion of FIG. 2, enlarging the undercut on the bottom surface of the frame.

FIG. 4 shows top perspective view of the frame of FIG. 1, with the dotted circled portion depicting an edge on the frame.

FIG. 5 shows an exploded view of the circled portion of FIG. 4, describing an edge on the frame.

FIG. 6 shows a bottom view of the frame of FIG. 1, with the dotted circled portion depicting one breaking feature on the frame.

FIG. 7 shows a perspective view of a multi-well plate, showing a frame portion as shown in FIG. 1 and tubes or wells inserted therein, according to an example embodiment.

FIG. 8 shows front view of the multi-well plate of FIG. 7, with the dotted circled portion depicting an undercut on the bottom surface of the frame.

FIG. 9 shows an exploded view of the dotted circled portion of FIG. 8, describing the undercut on the bottom surface of the frame.

FIG. 10A shows a cross section view of a tube/well interlocking into a frame and depicts a portion of the frame interlocking with a portion on the tube/well.

FIG. 10B shows a perspective view of a tube/well, depicted without a frame, and shows frame interlocking portions including a chimney and a lug.

FIG. 10C shows a cross-section view of a tube/well, depicted without a frame, and shows frame interlocking portions including a chimney and a lug.

FIG. 11 shows top perspective view of the multi-well plate of FIG. 7, with the circled portion depicting an edge on the frame portion thereof.

FIG. 12 shows an exploded view of the dotted circled portion of FIG. 11, describing an edge on the frame portion thereof.

FIG. 13 shows bottom view of the multi-well plate of FIG. 7, depicting an array of segmented breaking features in columnar arrangement thereon.

FIG. 14 shows a perspective view of a multi-well plate as shown in FIG. 7 with one tube-strip or well-strip being torn away therefrom, according to an example embodiment.

FIG. 15 depicts a perspective view if one tube strip or well-strip that has been torn or divided out of a multi-well plate as shown in FIGS. 7 and 14, according to an example embodiment.

FIGS. 16A and 16B depicts a right side and a left side view respectively of the tube strip or well-strip of FIG. 15, according to an example embodiment.

FIGS. 17A and 17B depicts a top and a bottom view respectively of the tube strip or well-strip of FIG. 15, according to an example embodiment.

FIG. 18 shows a perspective view of a frame portion of a multi-well plate, according to another example embodiment.

FIG. 19 shows front view of the frame of FIG. 18, with the dotted circled portion depicting an undercut on the bottom surface of the frame.

FIG. 20 shows an exploded view of the dotted circled portion of FIG. 19, describing the undercut on the bottom surface of the frame.

FIG. 21 shows top perspective view of the frame of FIG. 18, with the circled portion depicting an edge on the frame.

FIG. 22 shows an exploded view of the dotted circled portion of FIG. 21, describing an edge on the frame.

FIG. 23 shows bottom view of the frame of FIG. 18, with the circled portion depicting one breaking feature on the frame.

FIG. 24 shows a perspective view of a multi-well plate showing a frame portion as shown in FIG. 18 and tubes or wells inserted therein, according to an example embodiment.

FIG. 25 shows front view of the multi-well plate of FIG. 24, with the circled portion depicting an undercut on the bottom surface of the frame.

FIG. 26 shows an exploded view of the dotted circled portion of FIG. 25, describing an undercut on the bottom surface of the frame.

FIG. 27A shows a cross section view of a tube/well interlocking into a frame and depicts a portion of the frame interlocking with a portion on the tube/well.

FIG. 27B shows a perspective view of a tube/well, depicted without a frame, and shows frame interlocking portions including a chimney and a lug.

FIG. 27C shows a cross-section view of a tube/well, depicted without a frame, and shows frame interlocking portions including a chimney and a lug.

FIG. 28 shows top perspective view of the multi-well plate of FIG. 24, with the dotted circled portion depicting an edge on the frame portion thereof.

FIG. 29 shows an exploded view of the dotted circled portion of FIG. 28, depicting an edge on the frame portion thereof.

FIG. 30 shows bottom view of the multi-well plate of FIG. 24, depicting an array of segmented breaking features in columnar arrangement thereon.

FIG. 31 shows a perspective view of a multi-well plate as shown in FIG. 24 with one tube-strip or well-strip being torn away therefrom, according to an example embodiment.

FIG. 32 depicts a perspective view if one tube strip or well-strip that has been torn or divided out of a multi-well plate as shown in FIG. 24 or FIG. 31, according to an example embodiment.

FIGS. 33A and 33B depicts a right side and a left side view respectively of the tube strip or well-strip of FIG. 32, according to an example embodiment.

FIGS. 34A and 34B depicts a top and a bottom view respectively of the tube strip or well-strip of FIG. 32, according to an example embodiment.

### DETAILED DESCRIPTION

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. Use of “or” means “and/or” unless stated otherwise. The term “and/or” means that the terms before and after can be taken together or separately. For illustration purposes, but not as a limitation, “X and/or Y” can mean “X” or “Y” or “X and Y”.

One aspect of the present disclosure provides a multi-well plate that is manually divisible into strips of wells or tubes that are compatible with reagent dispensing by human or automation systems in manufacturing environments. In some examples, the present multi-well plates are useful to produce kits for various biological or chemical processes, wherein the kit reagents comprised in a multi-well plate of the present disclosure, can be produced in a high-throughput environment and sold to low throughout consumers/users. Accordingly, multi-well plates of the disclosure, advantageously allow prevention of wastage of expensive reagents.

In some embodiments, multi-well plates of the disclosure, are compatible with one or more of the following including but not limited to: automation, manufacturing processes, lyophilization procedures, filling of reagents, freezing reagents, use in thermal cycling instruments, use in microbial processes and the like.

Multi-well plates of the disclosure can be used for reagent storage and/or downstream processing of reactions such as but not limited to nucleic acid amplification, nucleic acid sequencing, sample preparation to extract biomolecules of interest including nucleic acids, proteins, carbohydrates, lipids among others, growing and culturing microbes.

Some example embodiments described herein provide divisible multi-well plates that can be divisible in to 8-tube strips or segments of one, two, three or more 8-tube strips, as required by an end user. Multi-well plates of the disclosure and the corresponding 8-tube strips and/or segments comprising at least two or more 8-tube strips are compatible and therefore mechanically fit and interface with several commercially available thermal cycler instruments, nucleic acid sequencing devices, sample preparation devices, microbial incubators and other bioprocess devices.

In some embodiments, the present disclosure provides a multi-well plate comprising: a frame portion made of a first material, wherein the frame portion has a planar top surface having an array of a plurality of holes arranged in a columnar pattern on the top surface, the frame portion further comprising an array of segmented breaking features arranged in a columnar pattern alternating with the columnar pattern of the plurality of holes and a plurality of wells made of a second material, wherein the wells are placed into the plurality of holes on the top surface of the frame, wherein the multi-well plate is manually divisible along the center of the segmented breaking features into one or more columns of multi-well strips.

Multi-well plates of the disclosure, are manually divisible along the center of the segmented breaking features into a plurality of single columns of multi-well strips. Multi-well strips can also be divided into segments comprising at least two columns of multi-well strips.

In some embodiments, segmented breaking features of the multi-well plates of the disclosure, comprise a plurality of petal shaped cuts that extend from the top surface of the frame portion through a bottom surface of the frame and a plurality of undercuts on a bottom surface of the frame portion, arranged such that the undercuts alternate with the petal shaped cuts. In some embodiments, the undercuts are V-shaped undercuts.

In some embodiments of a multi-well plate of the disclosure, petal shaped cuttings on the top deck of the frame combined with an undercut on the bottom of the frame creates eight small breaking sections or breaking features on each side of a column of multi-well plate. The length and thickness of each breaking section is the same across the frame. This pattern of breaking sections results in a segmented breaking pattern.

Advantageously, the segmented breaking pattern/feature, in contrast to continuous break lines that are present in some existing multi-well plates, provides one or more of the following improvements: eliminates the need of a tool for dividing the multi-well plate, and/or allows for manual tearing of the multi-well plate into strips/segments, and/or minimizes sharp edges after breaking due to the radius of petal shape cuts and the localized breaking and/or reduces sharp, jagged edges after breaking, and/or results in smooth edges and/or results in reduced incidences of glove tearing and injury to users.

In some embodiments, a skirt feature is located extending from the edges along the frame of a multi-well plate either on parts of the frame or all along the frame. In some embodiments, the skirt further has an extended lip that wraps all around the perimeter of the skirt and frame of plate. The lip is also described in the art as the SBS (Society for Biomolecular Screening) lip or as the SLAS (Society for laboratory Automation and Screening) lip, which is according to the ANSI (American National Standards Institute) for micro-well plates.

Multi-well plates of the present disclosure, in some embodiments, are low profile plates, wherein the skirt is shorter to accommodate tubes/wells of lower volumes. In some embodiments, low-profile tubes or well have a maximum volume of 0.2 mL when fully filled. Typical height of low profile tubes is about 15.50±0.25 mm. In some embodiments, low profile multi-well plates of the disclosure have a skirt lip, which is an extension of the skirt and is located on the four corners of the frame of a multi-well plate.

In other embodiments, multi-well plates of the disclosure, are standard profile plates, wherein the height of the skirt is of standard height, which allows tubes/wells of standard height to fit into the frame. In such embodiments, the skirt has an extended lip (the SBS or SLAS lip) that that wraps all around the perimeter of the skirt and plate. Such plates, in some embodiments, allow standard height multi-well tubes to be associated with the frame. Standard height or standard profile wells or tubes have a maximum well volume of 0.3 mL when used with adhesive and heat seals. Typical height of standard profile tubes or wells is about 20.70±0.25 mm.

Both standard profile and low profile multi-well plates of the disclosure and their divisible strips and segments are useful in commercially available thermocycler instruments such as those used to perform polymerase chain reaction (PCR) and quantitative PCR (qPCR). In non-limiting examples, the present semi-skirted multi-well plates and strips and segments derived therefrom mechanically fit and interface with exemplary thermocycler instruments such as Applied Biosystems 7500, QuantStudio 3 QuantStudio 5, QuantStudio 6, QuantStudio 7, Applied Biosystems ProFlex, Veriti, SimpliAmp, among others. The multi-well plates of the disclosure also fit and interface with heat sealers and aluminum support racks.

Multi-well plates and strips of the present disclosure, in some embodiments, can fit and interface with other instruments such as sequencing instruments and platforms, sample preparation instruments and robotic platforms and the like.

Another advantage of the multi-well plates of the present disclosure is therefore provision of a consumable multi-well plate with or without one or more of a pre-aliquoted, a frozen and/or a lyophilized reagent that are usable with common thermocycler instruments and other commonly used instruments and devices available in the market. A single strip or a segment comprising more than one strip can be used variously with different thermal cyclers.

The embodiments described below are exemplary embodiments with reference to the drawings. Like reference numerals and characters in the drawings refer to like elements or equivalents. However, the disclosure is not to be construed in limitation to the drawings and specific embodiments which are mere examples of concepts. For example, while most of the drawings depict 96-well plates that have 8×12=96 tubes/wells arranged in rows and columns, one of skill in the art, in light of the teachings of the present disclosure, will realize that these teachings extend to multi-well plates having more or fewer wells/tubes such as but not limited to multi-well plates with 24, 48, 96, 384 or 1536 or more wells/tubes.

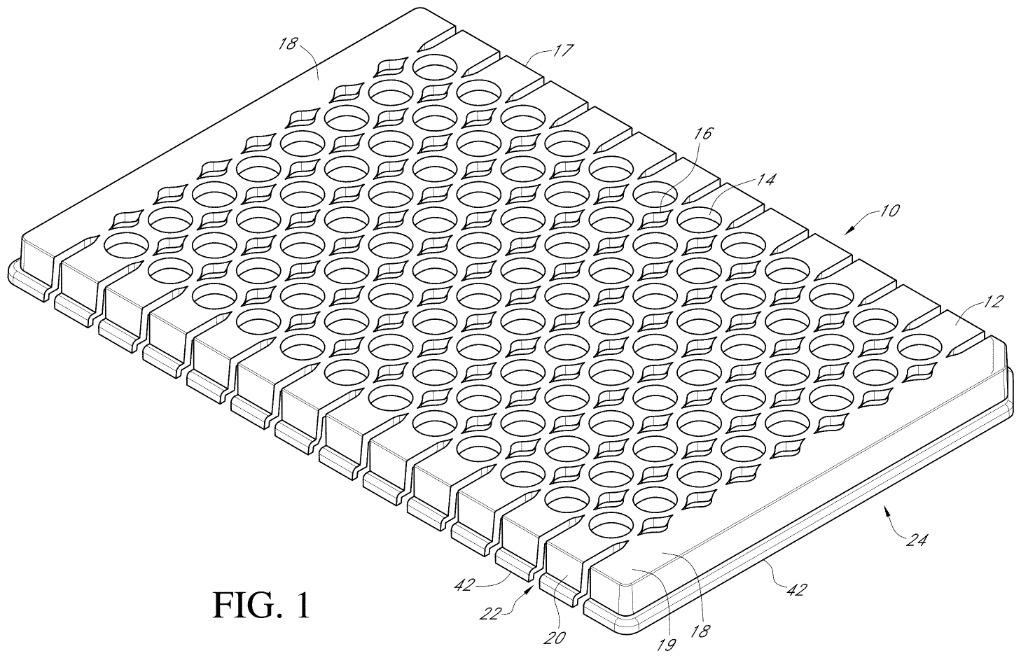

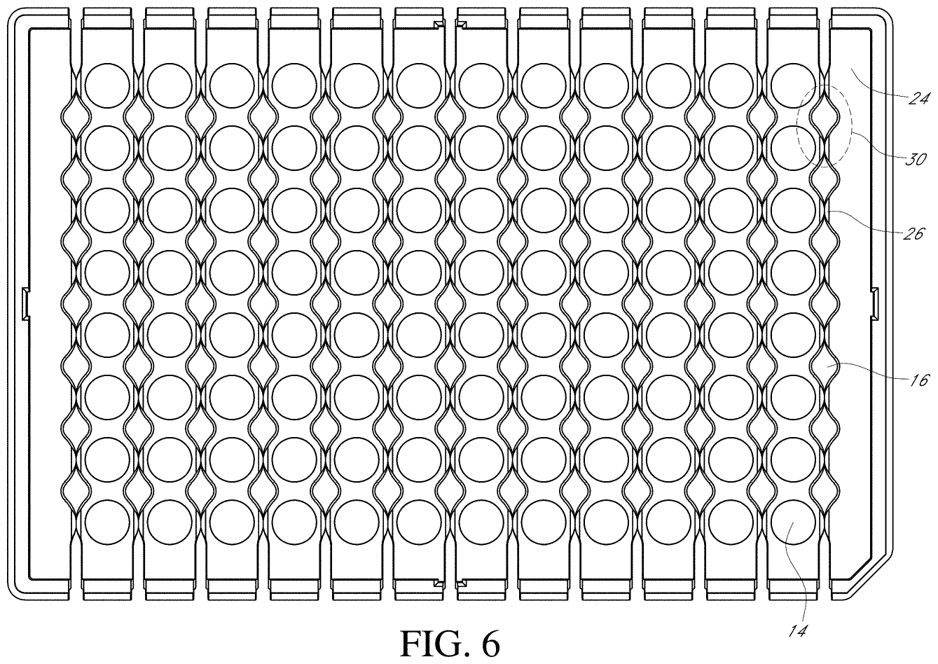

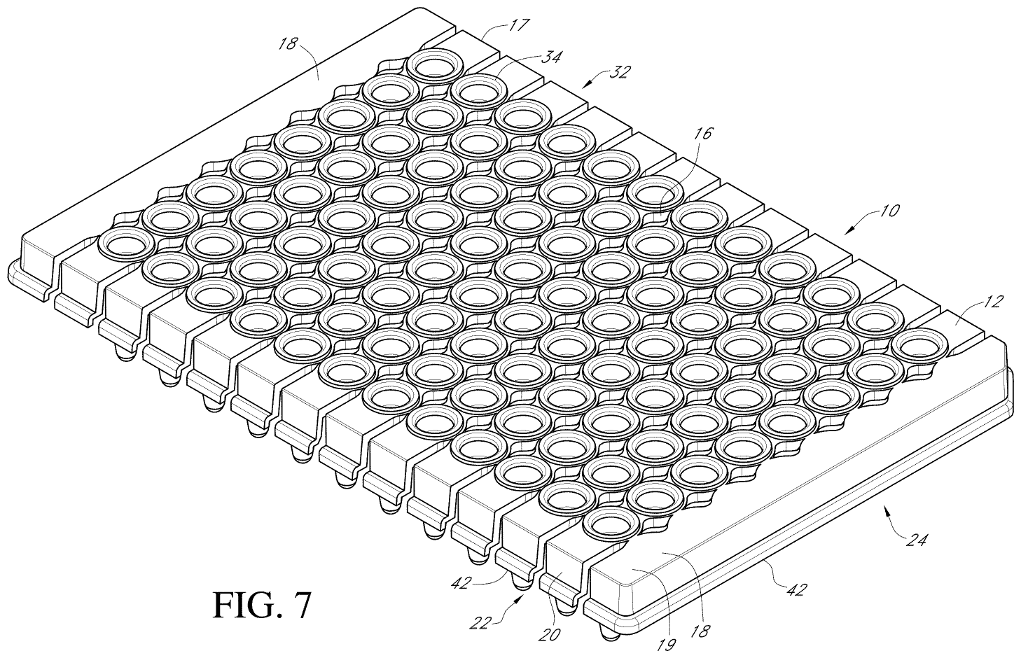

FIG. 1 shows a perspective view of a frame portion 10 of a multi-well plate according to an example embodiment. Frame 10 comprises a planar top surface 12 having an array of a plurality of holes 14 arranged in a columnar pattern on the top surface 12. Holes 14 are configured to receive therein tubes or wells 34 (tube/well 34 are depicted later, for example in FIG. 7). Frame portion 10 further comprises an array of petal shaped features 16 arranged in a columnar pattern alternating with the columnar pattern of the plurality of holes 14. Petal shaped features 16 are part of breaking features 30 described in further detail in sections below, for example see FIG. 6.

FIG. 1 also shows two edges 18 located on the left and right sides of the array of holes 14 and petal shaped features 16. Edges 18 can have alpha numeric labeling to mark or label holes 14, which are configured to receive therein tubes or wells 34 (tubes 34 are depicted in FIG. 7). In one non-limiting example, right edge 18 can have alphabetical numbering such as A, B, C, D, E, F, G, H, etc. Numeric labeling is also alternatively possible. Labeling may also be done on left edge 18.

FIG. 1 also shows top side 17 and bottom side 19 of the top planar surface 12 with a plurality of skirts 20 extending therefrom. A plurality of cuts 22 separate each column comprising holes 14 at the top side 17 and bottom side 19. Cuts 22 are aligned with petal shaped features 16 in a straight line. Columns comprising plurality of holes 14 are additionally defined on the two ends of the column by two cuts 22 can also be alphanumerically labelled on the top side 17 of FIG. 1.

In non-limiting examples, columns formed in between the plurality of cuts 22 can be labeled or marked on the top side 17 of frame 10 by numbers such as 1, 2, 3, 4, 5, 6, 7, 8, 9, 10, 11, 12, etc. These columns comprise a plurality of holes 14 arranged linearly. Alphabetical labeling is also alternatively possible for these columns. Labeling may also be dome on bottom side 19. Such labeling helps identify the tubes/wells and the reactions therein. For example, in the non-limiting labeling example above, a first tube 34 inserted on the top right side of frame 1 can be identified by the label A1.

FIG. 1 also depicts skirt lip 42, which according to this embodiment, wraps around the perimeter of the entire frame (with the exception of cuts 22). As can be seen skirt lip 42 extends from skirt 20. In this embodiment, skirt 20 is longer in height (as compared to another embodiment described later) and accommodate standard size tubes/wells. Hence frames with standard height skirt portions are described as standard profile multi-well plate frame. The embodiment of FIG. 1, with a skirt lip 42 wrapping all-around the perimeter of the plate frame (with the exception if cuts 22) is referred to as a skirted frame.

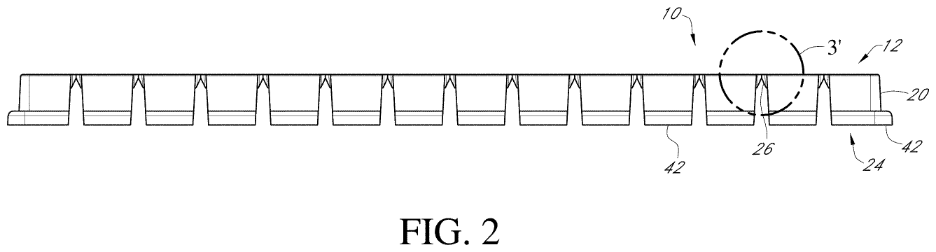

FIG. 2 shows a front view of frame portion 10 of a multi-well plate of the disclosure, wherein the circled portion depicts an undercut 26 located on the bottom surface 24 of frame 10. Undercut 26 and petal shaped features 16 are both comprised in breaking features 30 (shown in FIG. 6). Breaking features 30 are arranged as segmented breaking features.

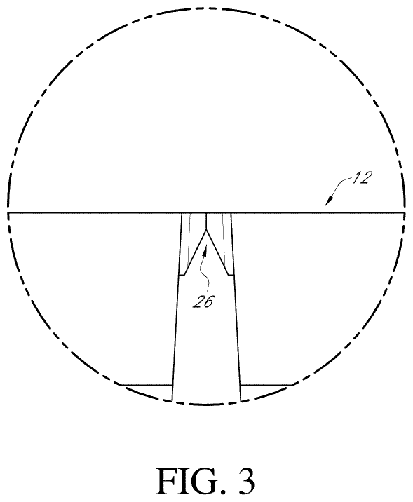

FIG. 3 shows an exploded view of the dotted circled portion of FIG. 2, describing undercut 26 located on bottom surface 24 of frame 10. In some embodiments, undercut 26 is V-shaped.

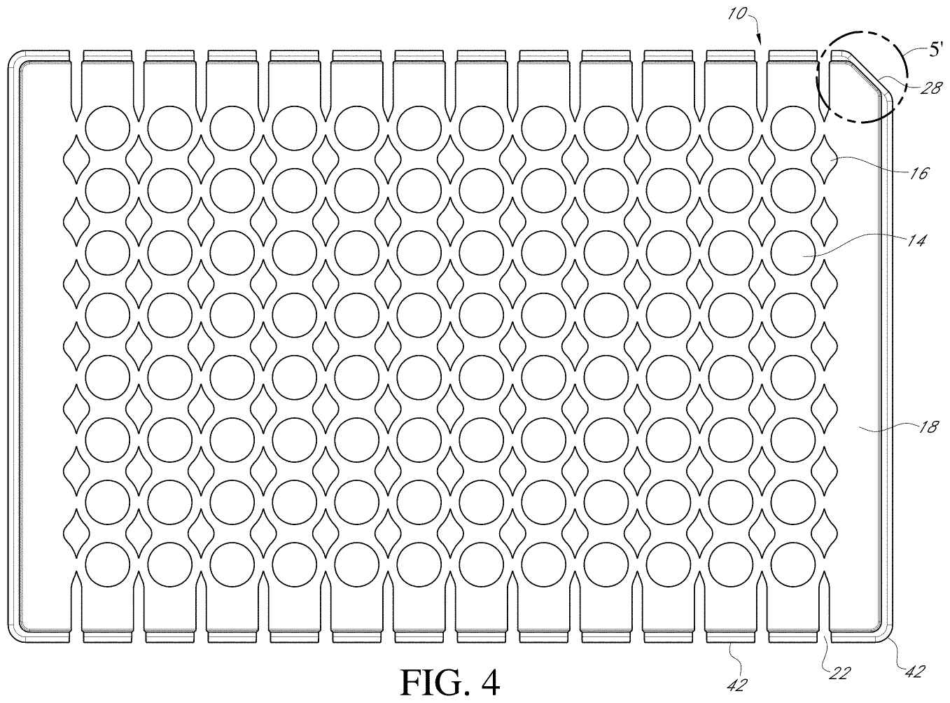

FIG. 4 shows a top perspective view of frame portion 10 of a multi-well plate, with the dotted circled portion depicting an edge 28 of frame 10.

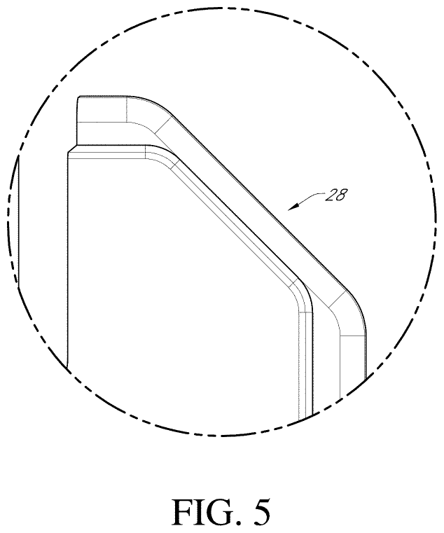

FIG. 5 shows an exploded view of the dotted circled portion of FIG. 4, describing the depicting an edge 28 on frame 10. Edge 28 on frame 10 can be used for plate orientation in instruments where the multi-well plates are used including automated filling instruments, PCR instruments and the like.

FIG. 6 shows bottom view of frame portion 10 of FIG. 1, with the dotted circled portion depicting one breaking feature 30 formed on the frame. As noted above, one breaking feature 30 comprises a petal shaped feature 16 and an undercut 26. A plurality of breaking features 30 are formed beginning from one cut 22 located on one end (e.g. tops side 17) of frame 10 and going along a linear path to another cut 22 located the opposite side of frame 10 (e.g., bottom side 19). This pattern forms a plurality of segmented breaking features 30. Segmented breaking features 30 allow for manual tearing with smooth edges, with no rough edges or with significantly reduced rough or jagged ends.

FIG. 7 shows a perspective view of top surface 12 of a multi-well plate 32 showing a frame portion 10 (of standard profile as shown in FIG. 1) and tubes or wells 34 inserted into holes 14 thereon, according to an example embodiment.

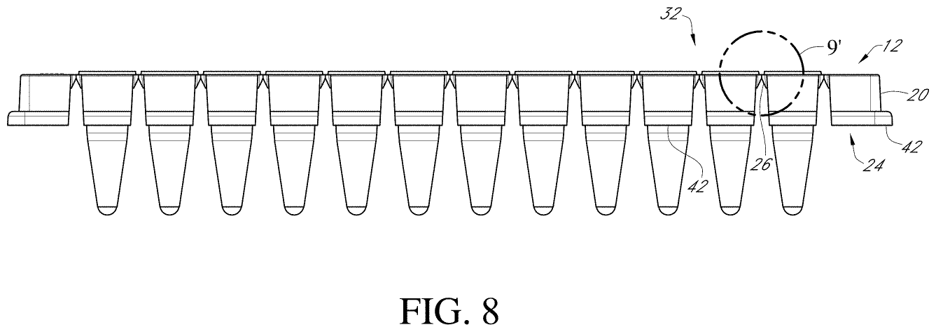

FIG. 8 shows front view of the multi-well plate of FIG. 7, with the dotted circled portion depicting an undercut 26 on the bottom surface 24 of the frame.

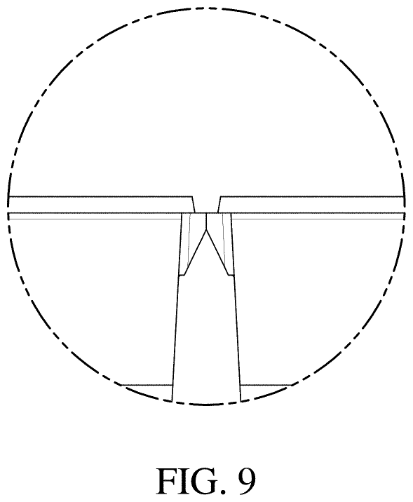

FIG. 9 shows an exploded view of the dotted circled portion of FIG. 8, describing undercut 26 on the bottom surface 24 of frame 10.

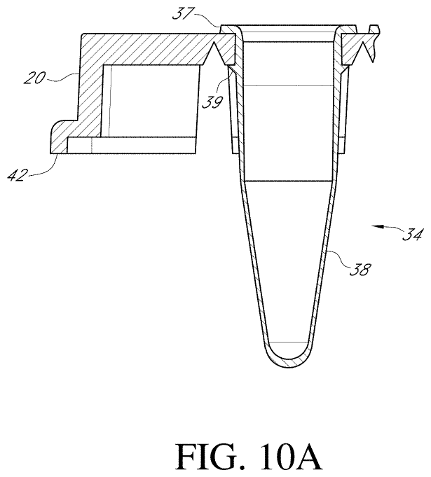

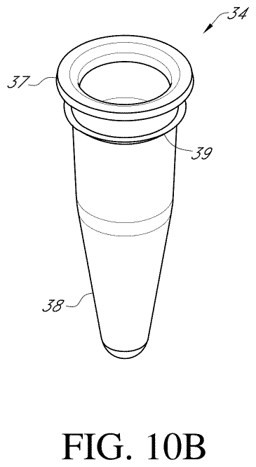

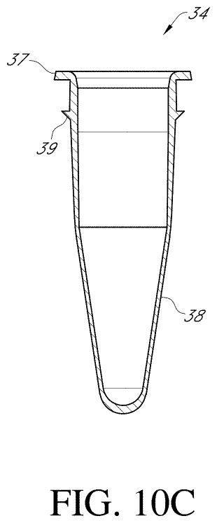

FIG. 10A shows a cross section view of a well or tube 34 interlocking into a frame 10 and depicts a chimney 37 of the frame 10 interlocking with a lug 39 located on the side of tube/well 34. Skirt 20 and lip 42 are also shown in this figure.

FIG. 10B shows a perspective view of tube 34, depicted without frame 10, and shows chimney 37 and lug 39.

FIG. 10C shows a cross-section view of tube/well 34, depicted without frame 10, and shows frame interlocking portions including a chimney 37 and a lug 39.

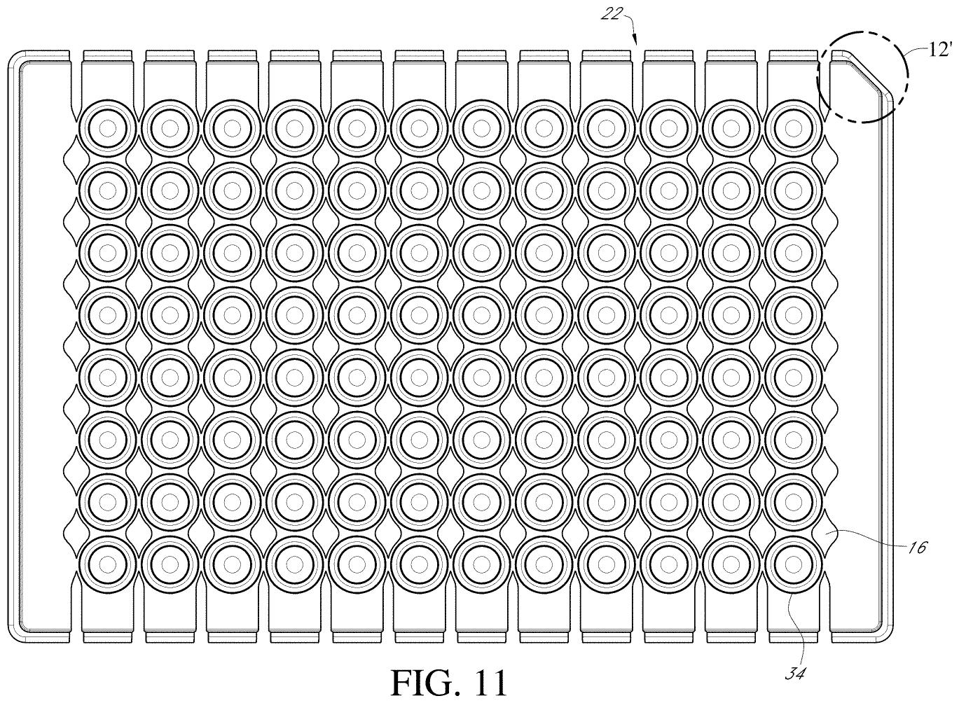

FIG. 11 shows top perspective view of the multi-well plate of FIG. 7, with the dotted circled portion depicting an edge 28 on frame portion 10 thereof.

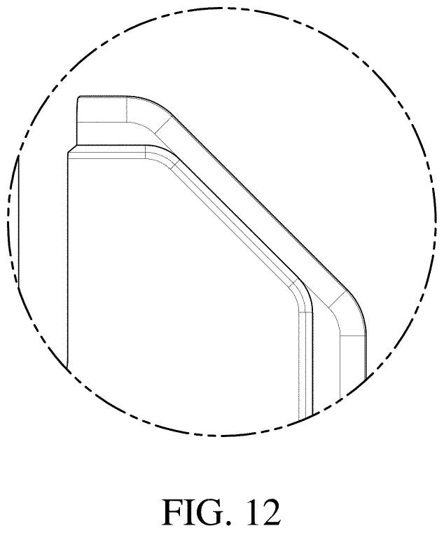

FIG. 12 shows an exploded view of the dotted circled portion of FIG. 11, describing the depicting edge 28 on frame portion 10 thereof.

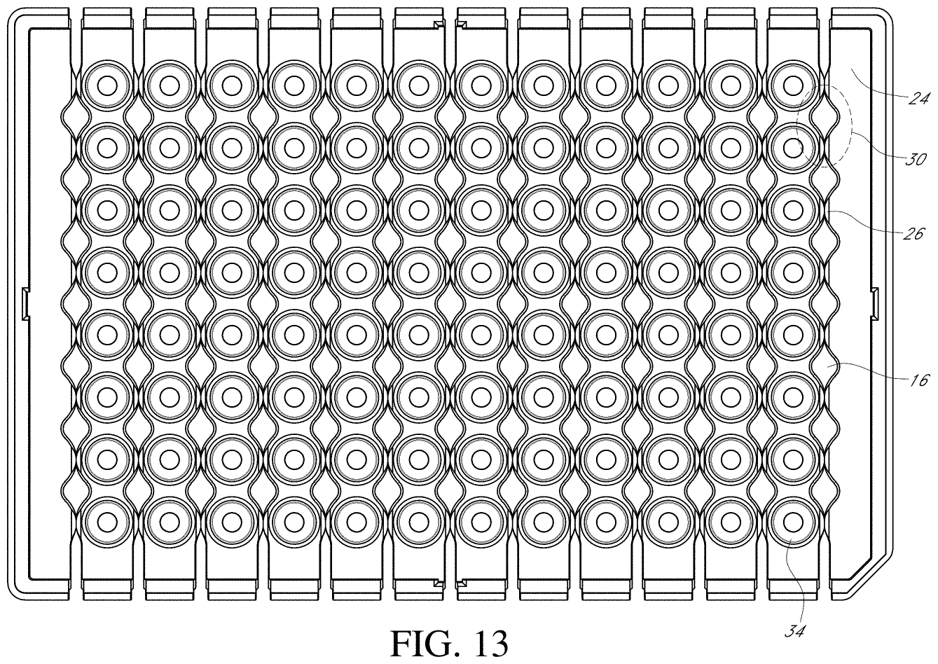

FIG. 13 shows bottom view of multi-well plate 32 of FIG. 7, having tubes 34 inserted in holes 14, depicting an array of segmented breaking features 30 in linear columnar arrangement on the bottom surface 24, extending between two cuts 22, wherein each cut 22 is located on opposite sides of each other on frame 10.

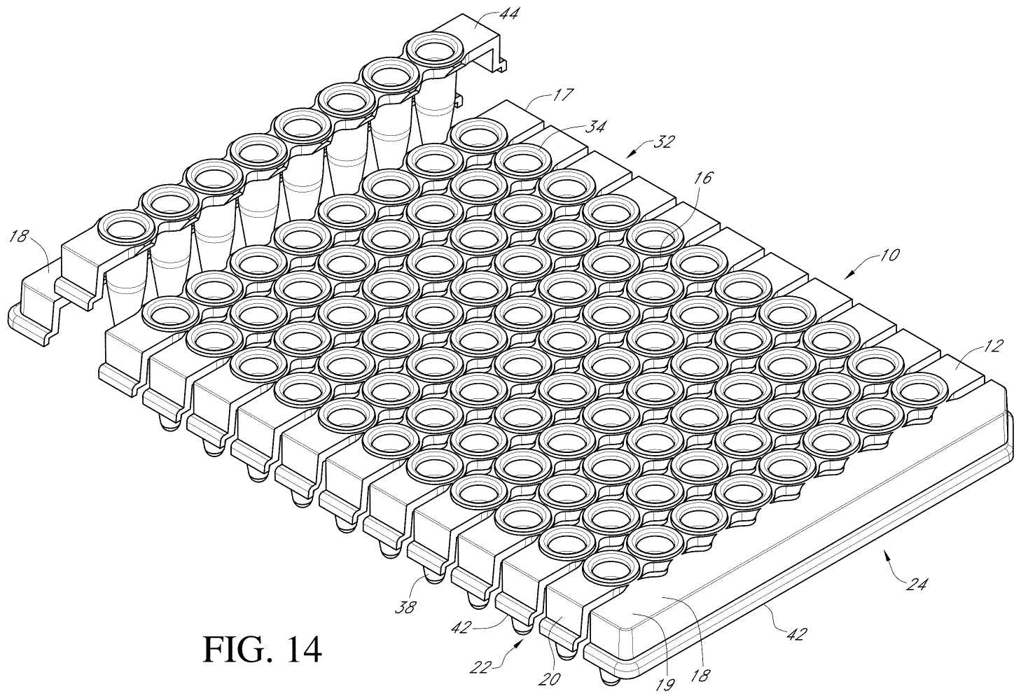

FIG. 14 shows a perspective view of a multi-well plate 32 as shown in FIG. 7 with one tube-strip or well-strip 44 being torn away therefrom, according to an example embodiment.

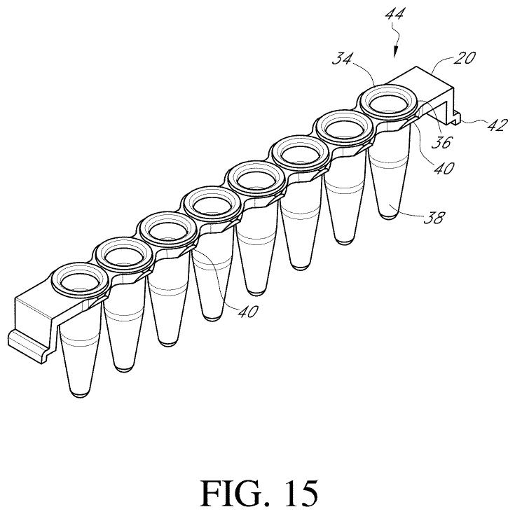

FIG. 15 depicts a perspective view of one tube strip 44 (also referred to herein alternatively as well-strip 44) that has been torn or divided out of a multi-well plate 32 as shown in FIGS. 7 and 14, according to an example embodiment. Tube-strip 44 depicts an exemplary top portion or rim 36 of a tube 34. 38 depicts bottom portion of tube 34 and a plurality if torn edges 40. In some embodiments, torn edge 40 is a smooth edge. In some embodiments, torn edge 40 is not jagged. In some embodiments, torn edge 40 is not rough. In some embodiments, torn edge 40 has significantly reduced sharp edges or significantly reduced jagged edges as compared to other divisible or tearable plates that are commercially available. Skirt 20 and skirt lip 42 are seen on the side edges of tube strip 44.

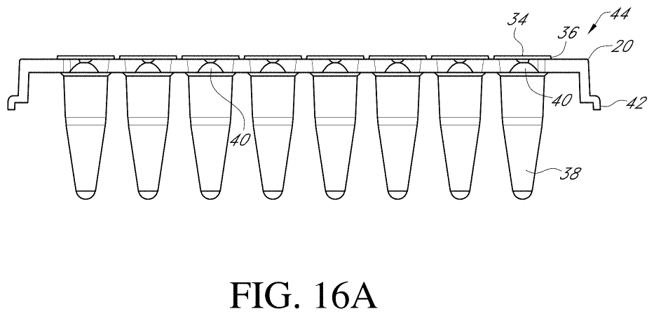

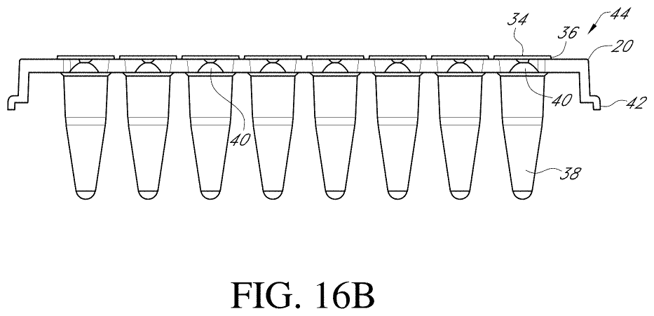

FIGS. 16A and 16B depict a right side and a left side view respectively of the tube strip 44 of FIG. 15, according to an example embodiment and show among other features torn edges 40, skirt lip 42 and skirt 20.

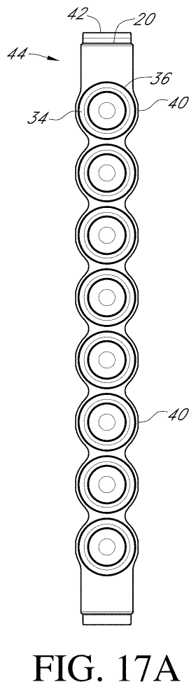

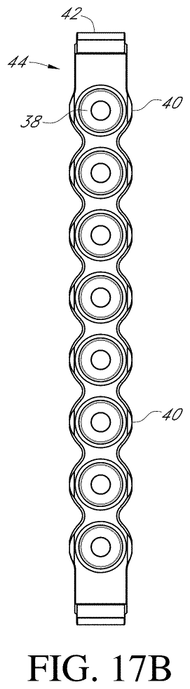

FIGS. 17A and 17B depict a top and a bottom view respectively of the tube strip or well-strip of FIG. 15, according to an example embodiment.

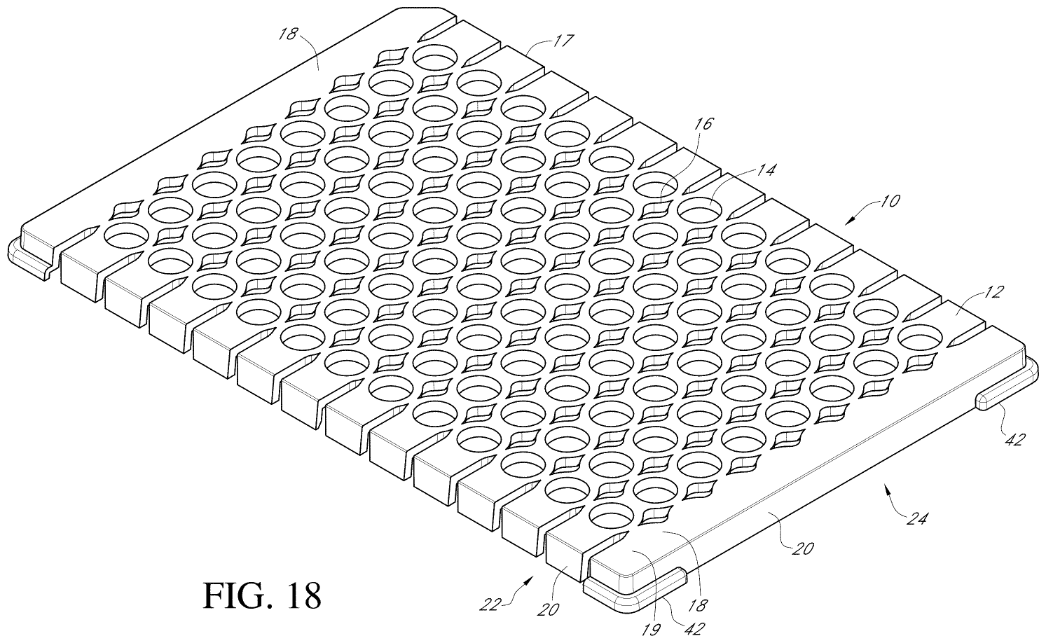

FIG. 18 shows a perspective view of a frame portion 10 of a multi-well plate 32, according to another example embodiment. FIG. 18 is very similar to FIG. 1, except that the skirt portions 20 are shorter and skirt lip portion 42 is located only on the four corners of plate 32. Embodiments where skirt 20 is shorter and lip 42 is only located at the corners are described as low profile plates since they have shorter skirts 20 as compared to standard profile plate skirts 20 shown for example in FIGS. 1 and 7. Low profile plates accommodate lower volume tubes as compared to standard profile plates.

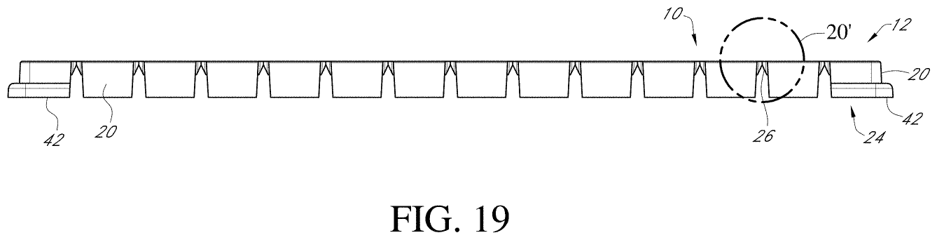

FIG. 19 shows front view of frame portion 10 of the low profile multi-well plate 32 of FIG. 18, with the circled portion depicting an undercut 26 on the bottom surface 24 of frame 10.

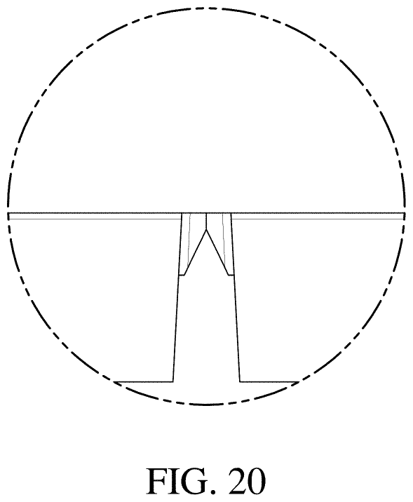

FIG. 20 shows an exploded view of the dotted circled portion of FIG. 18, describing undercut 26 on bottom surface 24 of frame 10.

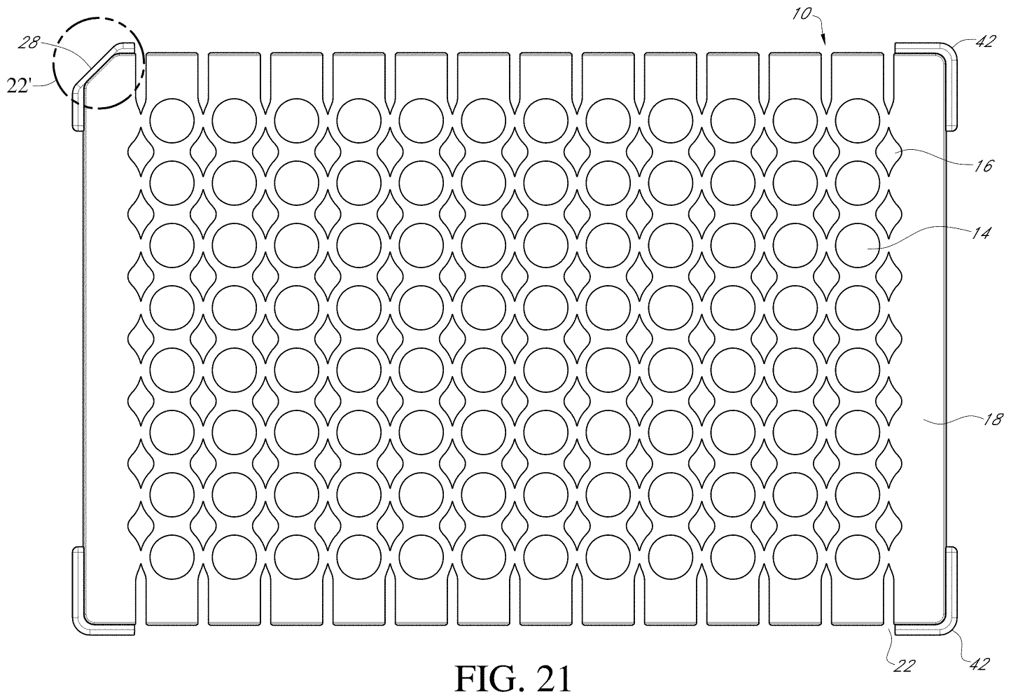

FIG. 21 shows top perspective view of frame portion 10 of multi-well plate 32 of FIG. 18, with the dotted circled portion depicting an edge 28 on the frame. Edge 28 is helpful for plate orientation in instruments.

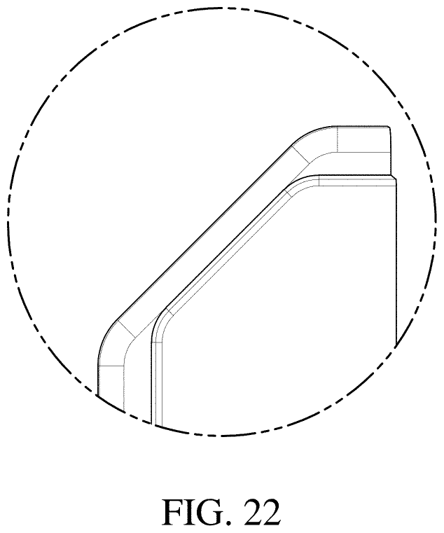

FIG. 22 shows an exploded view of the dotted circled portion of FIG. 21, describing the depicting edge 28 on frame 10.

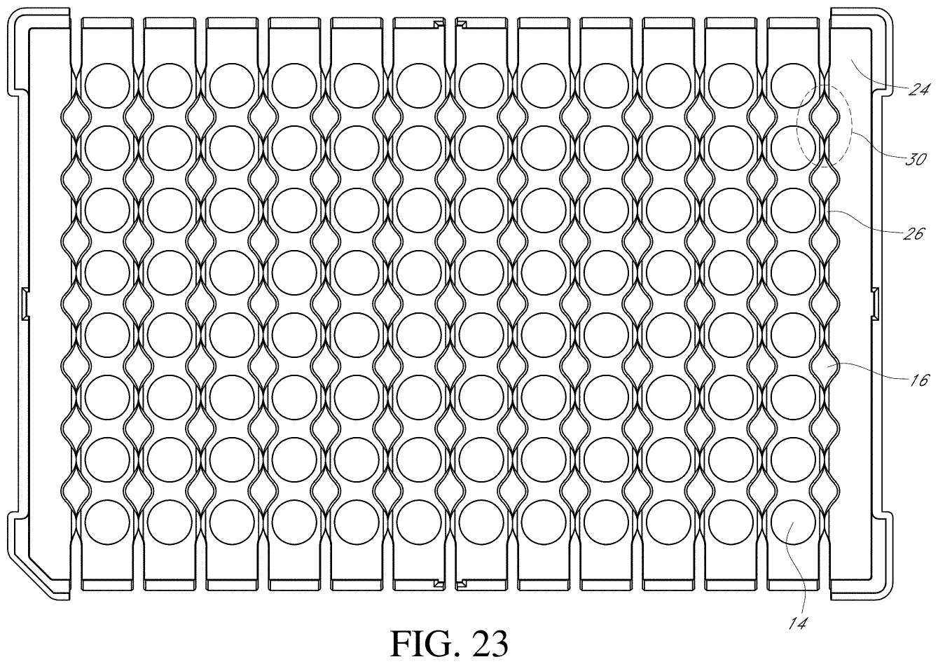

FIG. 23 shows bottom view of frame portion 10 of the multi-well plate 32 of FIG. 18, with the dotted circled portion depicting one breaking feature 30 on frame 10.

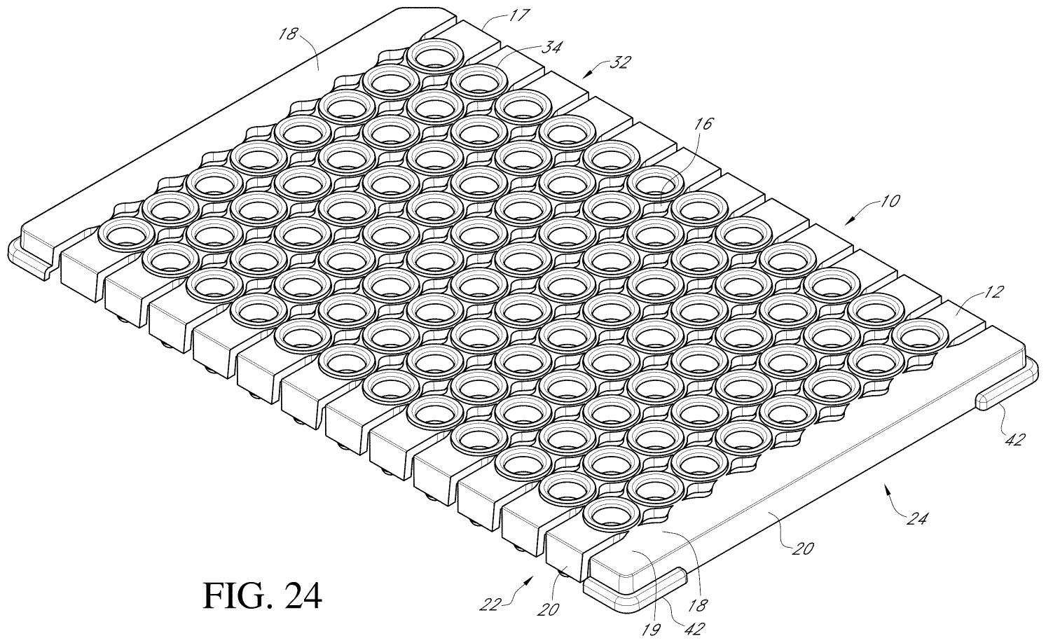

FIG. 24 shows a perspective view of a multi-well plate showing a low profile skirted 20 frame portion 10 as shown in FIG. 18 and tubes or wells 34 inserted in holes 14 located on top surface 12 of the plate, according to one example embodiment.

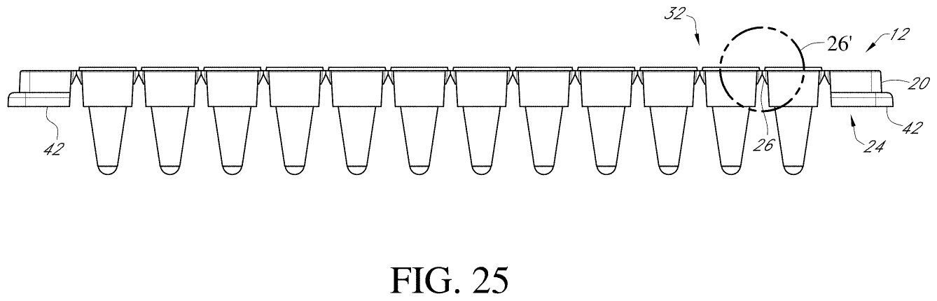

FIG. 25 shows front view of the multi-well plate 32 of FIG. 24, with the dotted circled portion depicting undercut 26 on bottom surface 24 of frame 10.

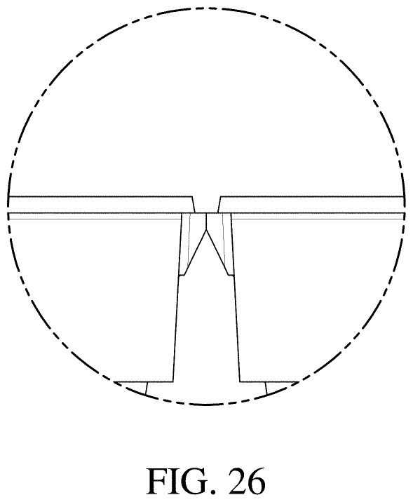

FIG. 26 shows an exploded view of the dotted circled portion of FIG. 25, describing undercut 26 on bottom surface 24 of frame 10.

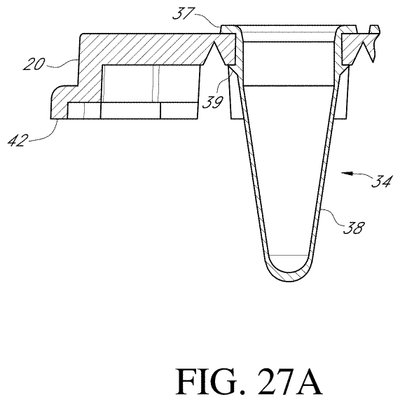

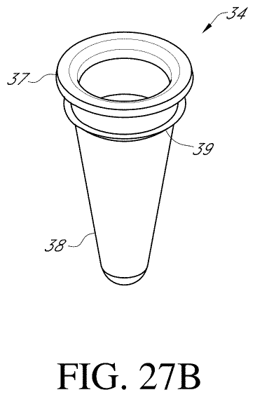

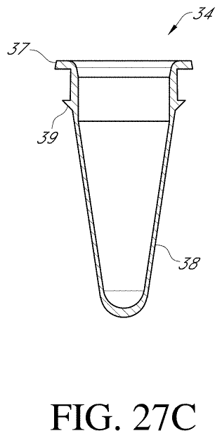

FIG. 27A shows a cross section view of a low-profile multi-well plate showing a tube/well 34 interlocking into frame 10 and further depicts a portion of the frame, referred to as chimney 37 interlocking with a portion on the tube/well referred to as lug 39.

FIG. 27B shows a perspective view of tube 34, depicted without frame 10, and shows chimney 37 and lug 39.

FIG. 27C shows a cross-section view of tube/well 34, depicted without frame 10, and shows frame interlocking portions including a chimney 37 and a lug 39.

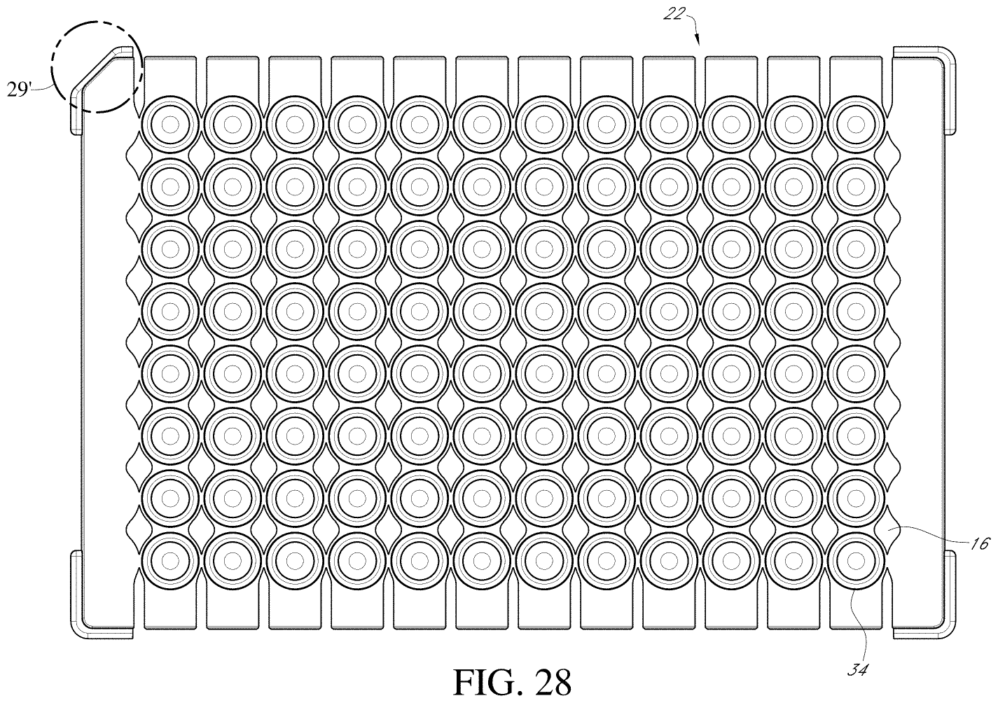

FIG. 28 shows top perspective view of the multi-well plate 32 of FIG. 24, with the dotted circled portion depicting an edge 28 on frame portion 10 thereof.

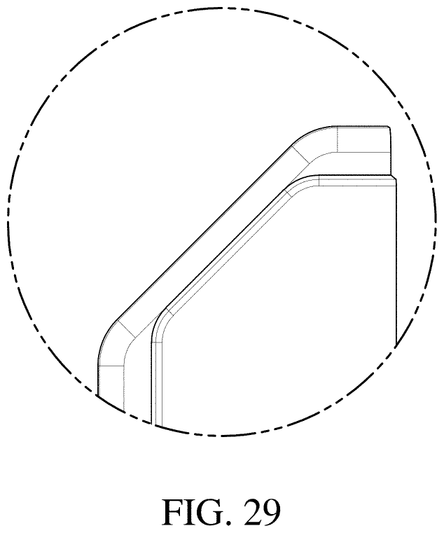

FIG. 29 shows an exploded view of the dotted circled portion of FIG. 28, describing edge 28 on frame portion 10 thereof.

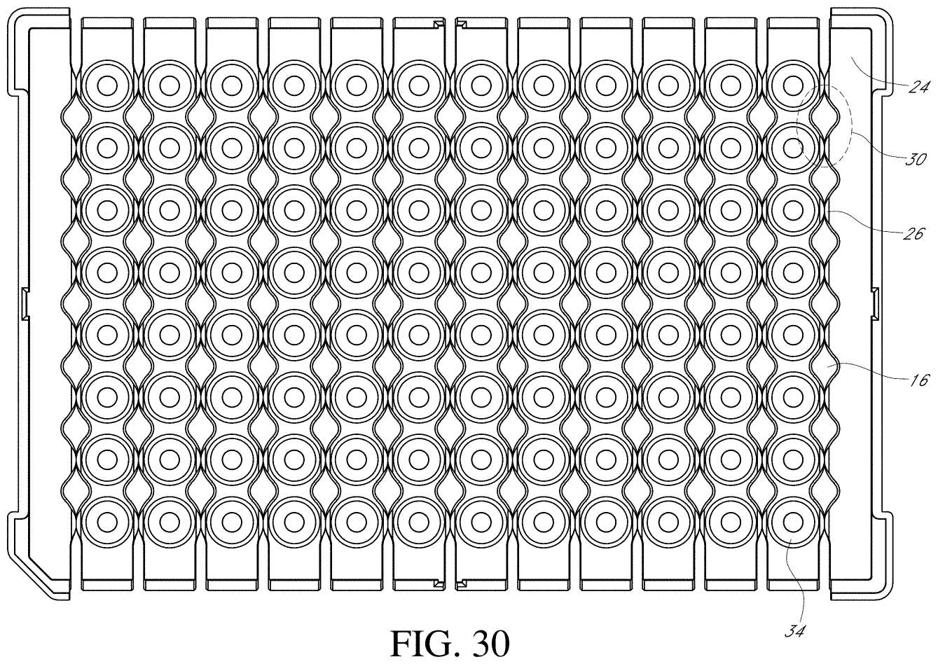

FIG. 30 shows bottom view of the multi-well plate 32 of FIG. 24, depicting an array of segmented breaking features 30 in a linear columnar arrangement thereon.

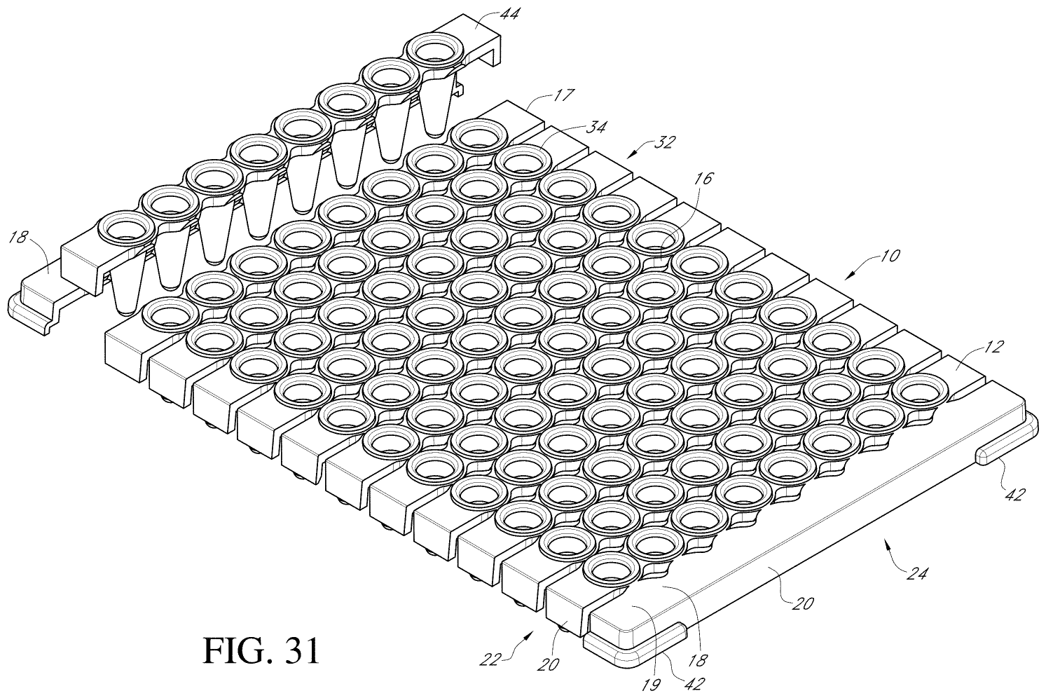

FIG. 31 shows a perspective view of a multi-well plate 32 as shown in FIG. 24 with one tube-strip 44 being torn away therefrom, according to an example embodiment.

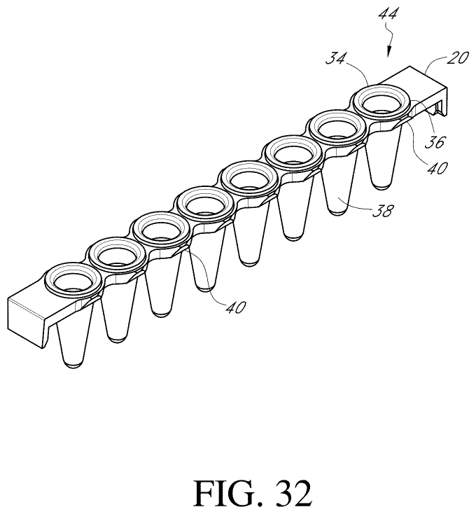

FIG. 32 depicts a perspective view of one tube strip 44 that has been torn or divided out of a multi-well plate 32 as shown in FIGS. 24 and 31, according to an example embodiment. This tube strip 44 is a low-profile tube strip, having a shorter skirt 20 and lacking a skirt lip portion since it is derived from a low-profile multi-well plate that only has the skirt lip 42 located on the four corners of the plate frame.

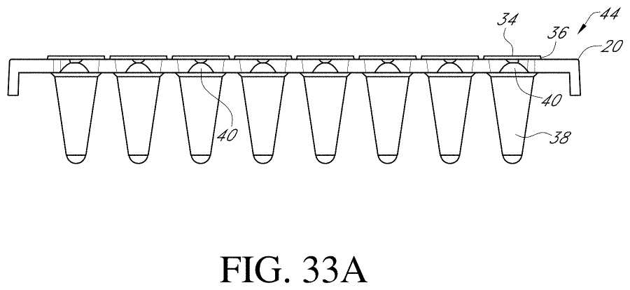

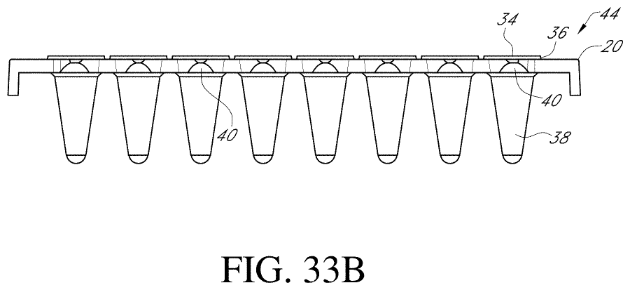

FIGS. 33A and 33B depicts a right side and a left side view respectively of the tube strip or well-strip 44 of FIG. 32, according to an example embodiment, and depicts a torn edge 40. In some embodiments, torn edge 40 is a smooth edge. In some embodiments, torn edge 40 is not jagged. In some embodiments, torn edge 40 is not rough. In some embodiments, torn edge 40 has significantly reduced sharp edges or significantly reduced jagged edges as compared to other divisible or tearable plates that are commercially available.

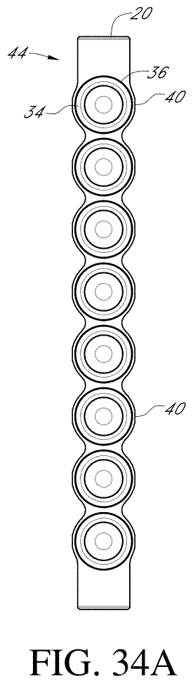

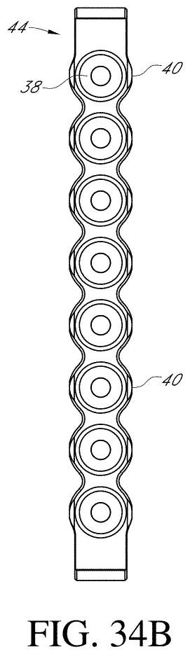

FIGS. 34A and 34B depicts a top and a bottom view respectively of the tube strip or well-strip 44 of FIG. 32, according to an example embodiment.

It will be appreciated by a person skilled in the art that numerous variations and/or modifications may be made to the present invention as shown in the specific embodiments without departing from the scope of the invention as broadly described. For example, the size and number of openings on the lid plate may be varied according to needs. The present embodiments are, therefore, to be considered in all respects to be illustrative and not restrictive.

1. A multi-well plate comprising:
a frame made of a first material, wherein the frame has a planar top surface having an array of a plurality of holes arranged in a columnar pattern on the top surface, the frame further comprising an array of segmented breaking features arranged in a columnar pattern alternating with the columnar pattern of the plurality of holes; and
a plurality of wells made of a second material, wherein the wells are placed into the plurality of holes on the top surface of the frame,
wherein the multi-well plate is manually divisible along a center of the segmented breaking features into one or more columns of multi-well strips.

2. The multi-well plate of claim 1, wherein the multi-well plate is manually divisible along the center of the segmented breaking features into a plurality of single columns of multi-well strips.

3. The multi-well plate of claim 1, wherein manually dividing the multi-well plate results in one or more columns of multi-well strips that have reduced sharp edges.

4. The multi-well plate of claim 1, wherein manually dividing the multi-well plate generates one or more columns of multi-well strips that have no sharp edges.

5. The multi-well plate of claim 1, wherein the segmented breaking feature comprises an array of petal shaped cuts that extend from the top surface of the frame through a bottom surface of the frame and a series of undercuts on a bottom surface of the frame portion, arranged such that the undercuts alternate with the petal shaped cuts.

6. The multi-well plate of claim 5, wherein the undercuts are V-Shaped undercuts.

7. The multi-well plate of claim 1, wherein a chimney located on the frame interlocks with a lug located on a side of the well.

8. The multi-well plate of claim 1, wherein the first material comprises polycarbonate and the second material comprises polypropylene.

9. The multi-well plate of claim 1, wherein the wells are directly moulded into the frame utilising form locking connections.

10. The multi-well plate of claim 1, wherein the frame further comprises a skirt that wraps all around the perimeter of the plate.

11. The multi-well plate of claim 1, wherein the frame further comprises a skirt that is located at the four corners of the plate.

12. The multi-well plate of claim 1, comprising 24, 48, 96, 384 or 1536 wells.

13. The multi-well plate of claim 11, comprising 96 wells.

14. The multi-well plate of claim 11, wherein the plate can be manually divided into one or more columns of 8 tube strips or into segments comprising at least one column of 8-tube strips.
